(19) United States
(12) Patent Application Publication
(10) Pub. No.: US 2025/0312115 A1
(43) Pub. Date: Oct. 9, 2025

(54) TECHNIQUES FOR CONTROLLING A COMPUTER-ASSISTED SYSTEM

(71) Applicant: Intuitive Surgical Operations, Inc., Sunnyvale, CA (US)

(72) Inventor: Ashwinram Suresh, San Jose, CA (US)

(73) Assignee: Intuitive Surgical Operations, Inc., Sunnyvale, CA (US)

(21) Appl. No.: 18/864,631

(22) PCT Filed: May 11, 2023
(86) PCT No.: PCT/US2023/021912

§ 371 Date: Nov. 11, 2024

Related U.S. Application Data
(60) Provisional application No. 63/340,573, filed on May 11, 2022.

(51) Int. Cl. A61B 34/37; A61B 90/00; G16H 40/63

(52) U.S. Cl. CPC A61B 34/37; G16H 40/63; A61B 2090/064

(57) ABSTRACT

A computer-assisted system includes a manipulator arm configured to support an instrument, an input device configured to accept user commands to move the instrument, and an actuator system coupled to the input device. A controller of the computer-assisted system is configured to determine a component of a change in force at the instrument, the component correlating with a direction of motion of the input device. The controller also determines a feedback force based at least in part on the environmental stiffness and the component of the change in force if the environmental stiffness experienced by the instrument exceeds a threshold stiffness. The controller causes the actuator system to drive the input device to apply the feedback force.

### CROSS-REFERENCE TO RELATED APPLICATIONS

This application claims the benefit of priority under 35 U S.C. § 119 (e) to U.S. Provisional Patent Application Ser. No. 63/340,573, filed on May 11, 2022, which is hereby incorporated by reference herein in its entirety.

### FIELD

Disclosed embodiments are related to computer-assisted systems such as robotic systems, techniques for controlling computer-assisted systems, and related methods of use.

### BACKGROUND

Computer-assisted electronic devices are being used more and more often. This is especially true in industrial, entertainment, educational, and other settings. As a medical example, the medical facilities of today have large arrays of electronic devices being found in operating rooms, interventional suites, intensive care wards, emergency rooms, and/or the like. Many of these electronic devices may be capable of autonomous or semi-autonomous motion. It is also known for personnel to control the motion and/or operation of electronic devices using one or more input devices located at a user control system. As a specific example, minimally invasive, robotic teleoperated systems permit medical personnel to teleoperate robotic systems to perform procedures on patients from a location within touching distance of the patient, from a location away from the patient but still within viewing distance of the unaided human eye, or in remote locations outside of the same facility, city, or country of the patient. Telesurgery refers generally to a teleoperated surgery performed using surgical systems where the surgeon uses some form of remote control, such as through commanding a servomechanism, to manipulate surgical instrument movements rather than directly holding and moving the instruments by hand.

### SUMMARY

In some embodiments, a computer-assisted system includes a manipulator arm configured to support an instrument, an input device configured to accept user commands to move the instrument, an actuator system coupled to the input device, and a controller comprising at least one processor. The controller is configured to determine a component of a change in force at the instrument, where the component correlates with a direction of motion of the input device. The controller is also configured to, in response to a determination that an environmental stiffness experienced by the instrument exceeds a threshold stiffness, determine a feedback force based at least in part on the environmental stiffness and the component of the change in force, and cause the actuator system to drive the input device to apply the feedback force.

In some embodiments, a method of controlling a computer-assisted system configured to support an instrument includes the following. The computer-assisted system comprises an input device configured to control movement of the instrument, and an actuator system configured to apply force to the input device. The method comprises determining a component of a change in force at the instrument, the component correlating with a direction of motion of the input device. The method also comprises in response to a determination that an environmental stiffness experienced by the instrument exceeds a threshold stiffness, determining a feedback force based at least in part on the environmental stiffness and the component of the change in force, and causing the actuator system to drive the input device to apply the feedback force.

In some embodiments, a non-transitory computer-readable storage medium stores instructions that, when executed by at least one processor associated with a computer-assisted device, causes the at least one processor to perform a method. The computer-assisted system is configured to support an instrument. The computer-assisted system comprises an input device configured to control movement of the instrument, and an actuator system configured to apply force to the input device. The method comprises determining a component of a change in force at the instrument, where the component correlates with a direction of motion of the input device. The method also comprises, in response to a determination that an environmental stiffness experienced by the instrument exceeds a threshold stiffness, determining a feedback force based at least in part on the environmental stiffness and the component of the change in force, and causing the actuator system to drive the input device to apply the feedback force.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a diagram of a computer-assisted system according to some embodiments;

FIG. 2 is block diagram of a computer-assisted system according to some embodiments

FIG. 3 is a diagram of a control loop for a computer-assisted system according to some embodiments;

FIG. 4 is a diagram of a control block for a computer-assisted system according to some embodiments;

FIG. 5 is a diagram of a portion of a control block according to some embodiments;

FIG. 6A depicts experimental results of an implementation of the control block of FIG. 5;

FIG. 6B depicts experimental results of an implementation of the control block of FIG. 5;

FIG. 7 is a diagram of a portion of a control block according to some embodiments;

FIG. 8A depicts experimental results of an implementation of the control block of FIG. 7;

FIG. 8B depicts experimental results of an implementation of the control block of FIG. 7;

FIG. 8C depicts experimental results of an implementation of the control block of FIG. 7;

FIG. 9 is a diagram of a portion of a control block according to some embodiments;

FIG. 10 is a simplified diagram of a computer-assisted system during operation according to some embodiments; and

FIG. 11 depicts a flow chart for a method of operating a computer-assisted system according to some embodiments.

### DETAILED DESCRIPTION

Stabilizing a computer-assisted system can sometimes be difficult, increase costs, require larger or more powerful actuators, decrease performance, introduce unfavorable feedback to a user of the computer-assisted system, and the like. For example, where an input device is employed as a “leader” to remotely control a “follower” manipulator arm, phase loss may be introduced between the input device and the manipulator arm. This phase loss may be due to physical separation of the input device from the manipulator arm, the associated time delay in sensing, responding to, and transmitting information, the time required for actuators to power and drive or transmission systems to move, physical imperfections in the system (e.g., backlash, etc.), and the like. In some cases where a leader input device of a computer-assisted system is used to facilitate teleoperation of a manipulator arm of the computer-assisted system, the phase loss may be significant and, can generate instabilities in the control of actuators of the input device and/or actuators of the manipulator arm. Further, the higher the loop gain used in the teleoperation control loop, the greater the likelihood of instabilities due to phase loss. As another example, instabilities in a control system for a computer-assisted system may also be caused by presence of resonance and anti-resonance in the mechanism. For example, input commands that match in frequency of the natural resonance mode of the manipulator can lead to growing response and make it unstable. As another example, instabilities in a control system for a computer-assisted system may also be caused by a change in operational operating environment for a manipulator arm. For example, a manipulator arm may support an instrument that, in some operating environments and instances, can engage with material such as objects that differ in rigidity or compliance. For example, the instrument may interact with one or more harder, more rigid surfaces of an object as well as one or more softer (e.g., less hard), more compliant (e.g., less rigid) surfaces of an object. As another example, the instrument may interact with harder, more rigid bulk of an object in an environment as well as softer (e.g., less hard), more compliant (e.g., less rigid) bulk of an object. As yet a further example, the instrument may interact with material in the environment that is not solid, such as liquid or gel-type materials. As used herein the hardness of an object may refer to an object's resistance to surface deformation and rigidity may be a measure of an object's or other material's resistance to bulk deformations. The change in the encountered environmental rigidity or compliance as the instrument interacts with these different surfaces, or bulks, or non-solid materials may also introduce instabilities in controlling actuators of an input device to provide feedback to the user due a rapid increase in force at the instrument.

A first approach to address this problem includes increasing the damping applied by the system to the input device. However, greater damping may introduce or increase offset error (e.g., error in output value relative to desired value), reduce usability, and/or impair user experience, etc. For example, greater damping applied to the input device can increase the amount of force needed to move the input device or to keep the input device in motion, cause the input device to reduce in speed more quickly, decrease the time it takes for the input device to stop moving, and the like. “Force” is used here to indicate linear force, rotational force (also called “torque”) or a combination linear and rotational force. These effects may cause the input device to feel less responsive, heavier, slower, or more difficult to move to a user. A second approach to address this problem includes “relaxing” the feedback tracking of the input device to the manipulator arm, for example by introducing frequency filters to ignore high frequency signals that may be more likely to cause instabilities. This approach may lead to less accurate correspondence of position and/or velocity between the input device and the manipulator arm. The first and second approaches can also be combined.

In various instances, the technique described in this disclosure may provide one or more of the following benefits, alone or in combination with each other. A computer-assisted system, according to some embodiments, includes an input device controlled manipulator arm, and provides feedback to a user of the input device while operating with greater stability of the input device and/or improved tracking accuracy between the input device and manipulator arm. In some instances, the improved tracking accuracy can present sub-millimeter tracking errors. In some embodiments, the disclosed technique reduces the amount of damping applied to an input device with, and applies little or no damping (e.g. barely or not detectable based on human senses) while a manipulator arm teleoperated by the input device is not experiencing reaction forces from the operating environment (e.g. the manipulator arm or an instrument supported by the manipulator arm is not in contact with any materials, or in free space).

In some embodiments, the disclosed technique provides a controller configured to compensate for operating environment impedance encountered by a teleoperated manipulator with reduced likelihood of increasing a control loop gain drastically, which reduces the likelihood of unstable force feedback rendered at the input device. For example, contact with a substantially rigid surface (e.g., bone or another instrument in a surgical example) or substantially rigid bulk, or some other material of greater rigidity (e.g., a more viscous fluid, a stiffer gel, etc.) may cause a rapid increase in control loop gain if not compensated for because of a rapid increase in input force measured at the instrument. In some embodiments, the disclosed technique provides a controller configured to compensate for changes in the stiffness of the kinematic chain that comprise a teleoperated manipulator arm. For example, a fully extended manipulator arm may be more compliant and less stiff than a fully retracted manipulator arm, which may also result in increases in control loop gain if not compensated for. An extended manipulator arm will have a different bending stiffness because of its physical configuration compared to a retracted manipulator arm, such that a force applied at the tip of an extended manipulator arm is more able to flex the manipulator arm due to an increased lever arm. In some embodiments, the disclosed technique reduces the mass of user input devices, which can improve user convenience and user feel. However, the inventors have also appreciated that such low-mass input devices may increase loop gain and make the input device more susceptible to instability. Accordingly, the inventors have appreciated a controller configured to compensate for increases in feedback control loop gain for input device feedback forces caused by one or more sources using exemplary methods described herein.

As used herein “stiffness” is a ratio of change in force to change in displacement. For example, a stiffness factor K_{stiffness} may be defined as

$${K}_{stiffness} = Δ Force / Δ Displacement$$

where the ΔForce is the change in force experienced by an instrument supported by a manipulator arm and ΔDisplacement is the distance traveled by the supported instrument that resulted in the change in force is applied. Stiffness as determined by systems described herein may an approximation of a physical stiffness characteristic of an environmental object, which is traditionally defined as a ratio of force to deformation.

According to exemplary embodiments described herein, a controller may respond to an effective environmental stiffness experienced by an instrument supported by a manipulator arm. The effective stiffness may be due to interactions of the instrument with a surface, with or within viscous material, within the bulk of solid material, and/or internal instrument interactions. In some embodiments, the controller and exemplary methods described herein do not distinguish among the causes of the experienced stiffness. In some embodiments, the controller and exemplary methods described herein do not distinguish where forces are applied on the instrument. In other embodiments, a controller may distinguish the location and/or direction of forces applied on the instrument. For example, directionality of different forces may be employed to control the instrument and feedback forces differently in different degrees of freedom. According to some specific examples discussed herein, a total effective stiffness for the instrument is determined based on how a point controlled by teleoperation (e.g., commanded in response to input provided by an operator via an input device) responds when the instrument is moved within an instrument environment.

For example, a computer-assisted system may comprise an actuator system coupled to the manipulator arm, where the second actuator system is configured to drive motion of the instrument by driving motion of the manipulator arm, or by driving transmission elements of the manipulator arm. The controller may be configured to determine the environmental stiffness based on sensor signals received from environmental sensors, or based on motions of the instrument or second actuator system metrics in response to commands to move the instrument. As a specific example, a controller may be configured to determine the environmental stiffness based on an amount of force or torque associated with the second actuator system when driving motion of the instrument (e.g., an amount of force or torque provided by the second actuator system, an amount of force or torque experienced as reaction forces or torques by the second actuator system, a proxy for the amount of force or torque-such as through current or voltage proxies for electrically driven actuators, etc.). As another specific example, a controller may be configured to determine the environmental stiffness based on a resulting motion of the instrument when driving the motion of the instrument with the second actuator system. The environmental stiffness can be determined, for example, as a function of force and linear displacement and/or of torque and angular displacement (e.g., an amount of rotation about a reference axis). As a specific example, an estimate of the environmental stiffness can be calculated as a ratio of the change in force to linear displacement, or as a ratio of change in torque to angular displacement (e.g., amount of rotation about a rotational joint axis or some other reference axis), etc.

In a teleoperation example, a controller of a computer-assisted system is configured to command an actuator system to drive motion of an instrument in response to an operator input received at the input device. The motion of the instrument may be measured based a control point fixed relative to the instrument (e.g., at a tip of an end effector of the instrument, at a jaw rotational joint for an instrument with jaws, etc.) The controller can be configured to determine the environmental stiffness based on motion of the instrument (determined as motion of the control point) when the actuator system is driving the motion of the instrument. In some embodiments, multiple control points are used to command the motion of the instrument, and the stiffness is determined based on an aggregated motion of the multiple control points (e.g., a weighted or unweighted average, a weighted or unweighted sum, etc.). The environmental stiffness can be determined, for example, as a function of force and the linear displacement of a control point (or of an average of multiple control points) and/or of torque and the angular displacement of a control point (or of an average of multiple control points), similar to described above.

In some embodiments, a computer-assisted system includes a manipulator arm configured to support an instrument, and an input device configured to accept user commands to move the instrument by moving the manipulator arm. The computer-assisted system may also include an actuator system configured to apply feedback forces to the input device based on one or more forces (linear and/or rotational forces) sensed at the manipulator arm. For example, in the case of a tele-operated computer-assisted system, the actuator system may apply forces to an input device based on forces sensed at a manipulator arm teleoperated by the input device. In this manner, the actuator system may be employed to provide force-feedback to a user contacting the input device. The computer-assisted system may also include a controller, including one or more processors, configured to command the actuator system to control forces applied to the input device, where the controller includes at least one processor configured to execute programming instructions which cause the at least one processor to perform exemplary methods described herein.

In some embodiments, a method of controlling a computer-assisted system includes limiting feedback force rendered to an input device based on a determination of an environmental stiffness experienced by an instrument supported by a manipulator arm. The method may include determining a change in force (linear or rotational force) experienced at the instrument and a direction of motion (e.g., a velocity) of an input device used to command teleoperated motion of the instrument. The method may include determining a portion of the change in force that correlates with the direction of motion of the input device. That is, the method may include determining a component of the change in force that is projected onto the direction of motion. The method may also include determining an environmental stiffness of the instrument-operating environment interaction. The environmental stiffness may be determined by measuring force and displacement at the instrument. The method may include determining a feedback force based at least in part on the environmental stiffness and the component of the change in force. For example, in some embodiments, the environmental stiffness may be compared to a stiffness threshold which may form the basis of the feedback force determination. In some embodiments, the environmental stiffness exceeding the threshold stiffness may cause the feedback force to be reduced (e.g., as a fixed or variable gain reduction). The method may include causing the computer-assisted system to apply the determined feedback force to the input device (e.g., using one or more actuators of an actuator system).

In addition to the above, the inventors have appreciated the benefits of a method of controlling a computer-assisted system that includes determining and/or applying a feedback force based on a velocity of the input device. In particular, the inventors have appreciated the benefits of a method that includes disregarding any components of the change in force not correlating with a direction of motion of the input device. In some embodiments, a method that includes determining a feedback force may include determining non-zero change in force only in directions where the user input device has non-zero velocity. In other embodiments, a method that includes causing an actuator system to apply determined forces may include gating the application of change in forces that the change in forces only apply in directions where the user input device has non-zero velocity. Accordingly, depending on the particular embodiment, forces may be determined while accounting for the input device velocity, or determined forces may simply not be applied to the input device. Such an arrangement may allow the feedback forces to be rendered to the input device based solely on the input motion provided by a user. External disturbances on the instrument not correlated with input device movement may be effectively disregarded. Accordingly, resonance modes and instabilities generated by unexpected external disturbances at an instrument may be addressed by such a method.

According to exemplary embodiments described herein, a method may be described without specific reference to directionality. In some embodiments, determined feedback forces, environmental stiffnesses, and other parameters may be directional or assigned directionality. For example, feedback forces and stiffnesses may be associated with three non-parallel translational degrees of freedom (e.g., cartesian x, y, and z directions). In such embodiments, feedback forces may be determined in each of the degrees of freedom of an input device. In some embodiments, feedback forces and stiffnesses may be determined in rotational degrees of freedom (e.g., pitch, roll, and yaw directions). In some embodiments, feedback forces and stiffnesses may be determined in any number of translational and rotational degrees of freedom or any sub-combination of rotational and translational degrees of freedom. In some embodiments, the comparison may be a comparison of scalars associated which each degree of freedom for the manipulator arm. In some embodiments, a reference frame for cartesian directions may be different between an input device and a manipulator arm. That is, in a global reference frame, an x direction of the input device may not be parallel with an x direction of the manipulator arm. Rather, movements and forces of the input device in the x direction may be transformed to movements and forces of the manipulator arm in the x direction based on their respective local reference frames. In some embodiments the degrees of freedom of the input device may be parallel to the degrees of freedom of the manipulator arm in a global reference frame, as the present disclosure is not so limited.

In some embodiments, a method of controlling a computer-assisted system includes determining a feedback force to apply to the input device. In some embodiments, the determination of feedback force may be based on a saturation function which is limited by a maximum environment stiffness as measured at an instrument. In some embodiments, the method may include determining a saturation limit of the saturation function based on a measured environmental stiffness and a threshold stiffness (e.g., a maximum allowed stiffness given the design or expected operation of the system). The threshold stiffness may be set at any appropriate time, such as at system design, at system manufacture (such to account for manufacturing variations), with in-field calibration, and the like. The threshold stiffness may be a threshold which, when not exceeded, provides a level of system stability that meets performance criteria. In some embodiments, this saturation limit may be an upper saturation limit, capping the amount of change in feedback force that is applied to the input device. In some embodiments, the upper saturation limit may be determined by dividing the threshold stiffness by the environmental stiffness when the environmental stiffness exceeds the threshold stiffness. In some embodiments, when the environmental stiffness does not exceed the threshold stiffness, the saturation function may function as a passthrough, not limiting a determined change in feedback force. In some embodiments, the feedback force may be determined by accumulating (e.g., integrating) a resultant change in force after applying the saturation limit of the saturation function. In some embodiments, a saturation limit may be based on a stiffness threshold and an input device velocity. In some such embodiments, the velocity of the input device may be proportional to the saturation limit.

The inventors have also appreciated that the methods described above and herein may introduce some undesirable unintended performance characteristics of the input device. Accordingly, the inventors have appreciated the benefits of a method of controlling a computer-assisted device that addresses these unintended performance characteristics.

In some embodiments, methods according to exemplary embodiments described herein may introduce steady state force error in a feedback force rendered at an input device. For example, where a saturation limit is applied and limited the feedback force change to a user, there may be steady state error in the force output to the user once the input device stops moving. Additionally, methods according to exemplary embodiments described herein may not render the change in feedback force in the case of a sudden external force on the instrument or a sudden loss of force on the instrument. This is due to the input device not moving. As discussed previously, in some embodiments feedback force may be determined and rendered to an input device based on the presence of velocity of the input device. Accordingly, if the input device has zero velocity, positive or negative change of force may not be rendered to the input device. In some cases, force feedback may be employed by a user to guide the manipulator arm or accomplish a task, so in some cases the inventors have appreciated that it may be beneficial to eliminate or alleviate error in force rendering generated by methods according to exemplary embodiments herein.

In view of the above, the inventors have appreciated a method of controlling a computer-assisted system that provides steady state force recovery to allow accurate force to be rendered to the user over time. In some embodiments, a method of controlling the computer-assisted system may include determining a restoring force to apply to the input device to eliminate steady state error between the feedback force and the force measured at an instrument of the manipulator arm. However, so that the restoring force does not generate instability, the restoring force is applied in directions perpendicular to any direction of motion of the input device. Accordingly, the restoring force will not have a component of it that lines up with direction of non-zero input device velocity. The effect of this condition is that the restoring force is applied when the input device is held stationary or approximately stationary in one or more directions. In such a case, the restoring force may be applied to the input device without generating velocity that is correlated to the restoring force. In the case the input device is not held stationary or approximately stationary, restoring force application would create a non-zero velocity of the input device, thereby causing the application of restoring force to be canceled in the new direction of motion. In some embodiments, the restoring force may be superimposed on a determined feedback force. In some embodiments, the restoring force may be increased over time until the combined force applied to the input device is equal (after compensating for scaling) to the force measured at an instrument. In some embodiments, the restoring force may not be applied until a threshold time period has passed, which may be based on the particular application. In some embodiments, the restoring force may be applied at a linear rate, as exponential decay, or any other suitable force profile.

In the view of the above, the inventors have also appreciated a method of controlling a computer-assisted system that envelops feedback force to ensure feedback force does not exceed (after accounting for scaling) force at an instrument. The enveloping of feedback force may function as a gate which eliminates force being rendered at the input device regardless of the absence of force at the instrument. Without such envelopment, a force rendered at the input device while the input device velocity is zero would not change if the force at the instrument changes, due to the velocity dependence of the determined feedback force to apply to the input device according to exemplary embodiments described herein. The force envelopment may be applied in all degrees of freedom of the input device, regardless of velocity, as the envelopment may only reduce force, not add to force. It should be noted that the force envelopment may apply to specific feedback forces according to exemplary embodiments described herein. In some embodiments, feedback forces may exceed (after accounting for scaling) forces measured at an instrument due to other implemented control loop methods.

In view of the above, the inventors have also appreciated a method of controlling a computer-assisted system that employs excess budget of a saturation limit of a saturation function to reduce or eliminate steady state error in force caused by exemplary methods described herein. In particular, the inventors have appreciated the benefits of a switch in a control loop that allows the control loop to compensate for a divergence between feedback force rendered at an input device and force measured at an instrument. In some embodiments, a method may include determining a deficit in a saturation limit and employing the determined deficit to ratchet down the diverge in force in a direction opposite that of the user input velocity. As the total change in force is still within the overall saturation limits due to accounting for the deficit, stability is maintained despite the increase in force. In some embodiments, a method may include determining a change in force, adding a previous error signal change in force from a control loop, and determining if the combined change of force is larger than the originally determined change in force. This method may also include determining if the change of force is in a direction opposing a direction of a velocity of the input device. In some embodiments, the determination may be based on determining if a component of the change of force is directly opposite a direction of velocity in a reference frame of the input device (e.g., in Cartesian space or joint space). Upon determining the change of force is in the direction opposing the direction of velocity of the input device, the method may include using the combined change of force as an input to a saturation function. Such a method may ensure the larger force is input to a saturation function under the condition where a change in force is opposing a direction of velocity of the input device.

According to exemplary embodiments described herein, force and velocities of a manipulator arm and input device may be employed to determine a feedback force to be applied to an input device. In some cases, a manipulator arm and input device may have different scales, such that when directly compared, a force or velocity of one may be much greater than a force or velocity of the other. For example, a manipulator arm may move a distance on the order of 1 m, whereas an input device may move a distance on the order of 10 cm. Accordingly, when directly compared, the manipulator arm velocity may be an order of magnitude greater than the input device velocity. Thus, in some embodiments, forces and velocities of the manipulator arm and input device may be scaled such that they may be appropriately compared. In some embodiments, such scaling may be based on a scaling factor of position tracking between the input device and the manipulator arm. Any suitable scaling factor for the velocities and forces may be employed, as the present disclosure is not so limited.

According to exemplary embodiments described herein, a method of controlling a computer-assisted system includes determining and applying feedback force as a part of a control loop. In some embodiments, the control loop may operate at a frequency suitable to apply feedback forces in a target frequency band. In some embodiments, methods according to exemplary embodiments described herein may be employed to produce feedback forces at an input device in frequency ranges between 0 and 1000 Hz based on human tactile perception. In some embodiments, a speed of a control loop may determine an accumulation time period for a control loop cycle. In some embodiments, a change in force may be accumulated (e.g., integrated) as a part of a control loop over a period of 1 millisecond (ms) to 10 ms. A control loop may operate at any other appropriate range of frequencies depending on the particular computer-assisted application, as the present disclosure is not so limited in this regard.

This disclosure describes various devices, elements, and portions of computer-assisted devices and elements in terms of their state in three-dimensional space. As used herein, the term “position” refers to the location of an element or a portion of an element in a three-dimensional space (e.g., three degrees of translational freedom along cartesian x-, y-, and z-coordinates). As used herein, the term “orientation” refers to the rotational placement of an element or a portion of an element (three degrees of rotational freedom—e.g., roll, pitch, and yaw, angle-axis, rotation matrix, quaternion representation, and/or the like). As used herein, and for a device with repositionable arms and/or other repositionable structures, the term “proximal” for a kinematic chain refers to a direction toward the base of the kinematic chain, and “distal” refers to a direction away from the base along the kinematic chain.

As used herein, the term “pose” of a rigid body refers to the multi-degree of freedom (DOF) spatial position and orientation of a coordinate system of interest attached to the rigid body. In general, a pose includes a pose variable for each of the DOFs in the pose. For example, a full 6-DOF pose for a rigid body would include 6 pose variables corresponding to the 3 positional DOFs (e.g., x, y, and z) and the 3 orientational DOFs (e.g., roll, pitch, and yaw). A 3-DOF position only pose for a rigid body would include only pose variables for the 3 positional DOFs. Similarly, a 3-DOF orientation only pose for a rigid body would include only pose variables for the 3 rotational DOFs. Further, a velocity of the pose captures the change in pose over time (e.g., a first derivative of the pose). For a full 6-DOF pose, the velocity would include 3 translational velocities and 3 rotational velocities (also called “angular velocities”). Poses with other numbers of DOFs would have a corresponding number of velocities translational and/or rotational velocities.

Aspects of this disclosure are described in reference to computer-assisted systems and devices, which may include systems and devices that are teleoperated, remote-controlled, autonomous, semiautonomous, robotic, and/or the like. Further, aspects of this disclosure are described in terms of an implementation using a surgical system. Knowledgeable persons will understand, however, that inventive aspects disclosed herein may be embodied and implemented in various ways, including robotic and, if applicable, non-robotic embodiments and implementations. Implementations on surgical systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperated systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (without return to a human or animal anatomy) and for procedures on human or animal cadavers. Further, these techniques can also be used for medical treatment or diagnosis procedures that include, or do not include, surgical aspects.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

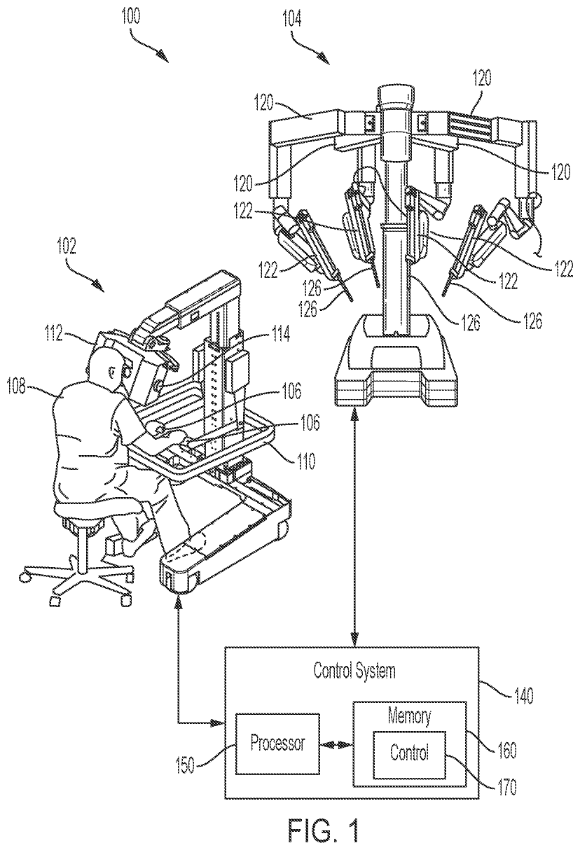

FIG. 1 is a simplified diagram of an example computer-assisted system, according to various embodiments. In some examples, the computer-assisted system is a teleoperated system 100. In medical examples, teleoperated system 100 can be a teleoperated medical system such as a surgical system. As shown, teleoperated system 100 includes a follower device 104 that may be teleoperated by being controlled by one or more leader input devices 106 configured to accepted external input, described in greater detail below. Systems that include a leader device and a follower device are referred to as leader-follower systems. As shown in FIG. 1 the input devices are a part of an input system that includes a workstation 102 (e.g., a console). In other embodiments, the input system can be in any appropriate form and may or may not include a workstation 102.

In the exemplary embodiment of FIG. 1, the workstation 102 includes one or more input devices 106 which are configured to be contacted and manipulated by a user 108. For example, the workstation 102 can comprise one or more input devices 106 configured for use by the hands, the head, or some other body part of user 108. Input devices 106 in this example are supported by workstation 102 and can be mechanically grounded. In some embodiments, an ergonomic support 110 (e.g., forearm rest) can be provided on which the user 108 can rest his or her forearms. In some examples, user 108 can perform tasks at a worksite during a procedure by commanding follower device 104 using input devices 106. In some embodiments, the workstation 102 may be physically located near the follower device 104. In other embodiments, the workstation 102 may be remotely located from the follower device 104, and may communicate with the follower device via one or more communication protocols over a local area network, wide area network, and/or via the internet.

As shown in FIG. 1, the workstation 102 includes a display unit 112. The display unit 112 can display images for viewing by user 108. In some embodiments, the display unit 112 can be moved in various degrees of freedom to accommodate the viewing position of user 108 and/or to optionally provide control functions as another input device. In the example of teleoperated system 100, displayed images can depict a worksite at which user 108 is performing various tasks by manipulating input devices 106 and/or display unit 112. In some examples, images displayed by display unit 112 can be received by workstation 102 from one or more imaging devices arranged at a worksite. In other examples, the images displayed by display unit 112 can be generated by display unit 112 (or by a different connected device or system), such as for virtual representations of tools, the worksite, or for user interface components.

According to the embodiment of FIG. 1, when using the workstation 102, user 108 can sit in a chair or other support in front of workstation 102, position his or her eyes in front of display unit 112, manipulate input devices 106, and rest his or her forearms on ergonomic support 110 as desired. In some embodiments, user 108 can stand at the workstation or assume other poses, and display unit 112 and input devices 106 can be adjusted in position (height, depth, etc.) to accommodate user 108.

In some embodiments, the one or more input devices 106 can be ungrounded (ungrounded input devices being not kinematically grounded, such as input devices held by the hands of user 108 without additional physical support). Such ungrounded input devices can be used in conjunction with display unit 112. In some embodiments, user 108 can use a display unit 112 positioned near the worksite, such that user 108 manually operates instruments at the worksite, such as a laparoscopic instrument in a surgical example, while viewing images displayed by display unit 112.

Teleoperated system 100 can also include follower device 104, which can be commanded by workstation 102. In a medical example, follower device 104 can be located near an operating table (e.g., a table, bed, or other support) on which a patient can be positioned. In some medical examples, the worksite is provided on an operating table, e.g., on or in a patient, simulated patient, or model, etc. (not shown). The follower device 104 shown includes a plurality of manipulator arms 120, each manipulator arm 120 configured to couple to an instrument assembly 122. An instrument assembly 122 can include, for example, an instrument 126. In various embodiments, one or more of instruments 126 can include an imaging device for capturing images (e.g., optical cameras, hyperspectral cameras, ultrasonic sensors, etc.). For example, one or more of instruments 126 could be an endoscope assembly that includes an imaging device, which can provide captured images of a portion of the worksite to be displayed via display unit 112. One or more instruments 126 can also include instruments configured to function in an operating environment, and physically interact with physical objects or characteristics of the operating environment (e.g., in a medical example, an operating environment may be a work space within a patient). Examples of potential instruments 126 may include, and are not limited to, graspers, scalpels, staplers, imagers or other sensors, cautery instruments, suction irrigators, and scissors. Any suitable instrument may be employed with a manipulator arm 120, as the present disclosure is not so limited.

In some embodiments, the manipulator arms 120 and/or instrument assemblies 122 can be controlled to move and articulate instruments 126 in response to manipulation of input devices 106 by user 108, and in this way “follow” through teleoperation the input devices 106. This enables user 108 to perform tasks at the worksite using the manipulator arms 120 and/or instrument assemblies 122. Manipulator arms 120 and follower device 104 are examples of repositionable structures on which instruments such as manipulating instruments or and/or imaging instruments including imaging devices can be mounted. The repositionable structure(s) of a computer-assisted system comprise the repositionable structure system of the computer-assisted system. For a surgical example, the user 108 could direct follower manipulator arms 120 to move instruments 126 to perform surgical procedures at internal surgical sites through minimally invasive apertures or natural orifices. The manipulator arms 120 and/or instrument assemblies 122 may also provide feedback information that is rendered to the user 108 through the one or more input devices 106. For example, in some embodiments, the manipulator arms 120 and/or instrument assemblies 122 may provide force information which is employed to apply feedback forces at the one or more input devices 106.

As shown, a control system 140 is provided external to workstation 102 and communicates with workstation 102. In other embodiments, control system 140 can be provided in workstation 102 or in follower device 104. As user 108 moves input device(s) 106, sensed spatial information including sensed position and/or orientation information is provided to control system 140 based on the movement of input devices 106. Control system 140 can determine or provide control signals to follower device 104 to control the movement of manipulator arms 120, instrument assemblies 122, and/or instruments 126 based on the received information and user input. In one embodiment, control system 140 supports one or more wired communication protocols, (e.g., Ethernet, USB, and/or the like) and/or one or more wireless communication protocols (e.g., Bluetooth, IrDA, HomeRF, IEEE 1002.11, DECT, Wireless Telemetry, and/or the like). In some embodiments, the control system 140 may be configured to implement exemplary methods herein and determine feedback forces to be applied to one or more input devices 106.

Control system 140 can be implemented on one or more computing systems. One or more computing systems can be used to control follower device 104. In addition, one or more computing systems can be used to control components of workstation 102, such as movement of a display unit 112.

As shown, control system 140 includes a processor 150 and a memory 160 storing a control module 170. In some embodiments, control system 140 can include one or more processors, non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, etc.), a communication interface (e.g., Bluetooth interface, infrared interface, network interface, optical interface, etc.), and numerous other elements and functionalities. In addition, functionality of control module 170 can be implemented in any technically feasible software and/or hardware.

Each of the one or more processors of control system 140 can be an integrated circuit for processing instructions. For example, the one or more processors can be one or more cores or micro-cores of a processor, a central processing unit (CPU), a microprocessor, a field-programmable gate array (FPGA), an application-specific integrated circuit (ASIC), a digital signal processor (DSP), a graphics processing unit (GPU), a tensor processing unit (TPU), and/or the like. Control system 140 can also include one or more input devices, such as a touchscreen, keyboard, mouse, microphone, touchpad, electronic pen, or any other type of input device.

A communication interface of control system 140 can include an integrated circuit for connecting the computing system to a network (not shown) (e.g., a local area network (LAN), a wide area network (WAN) such as the Internet, mobile network, or any other type of network) and/or to another device, such as another computing system.

Further, control system 140 can include one or more output devices, such as a display device (e.g., a liquid crystal display (LCD), a plasma display, touchscreen, organic LED display (OLED), projector, or other display device), a printer, a speaker, external storage, or any other output device. One or more of the output devices can be the same or different from the input device(s). Many different types of computing systems exist, and the aforementioned input and output device(s) can take other forms.

In some embodiments, control system 140 can be connected to or be a part of a network. The network can include multiple nodes. Control system 140 can be implemented on one node or on a group of nodes. By way of example, control system 140 can be implemented on a node of a distributed system that is connected to other nodes. By way of another example, control system 140 can be implemented on a distributed computing system having multiple nodes, where different functions and/or components of control system 140 can be located on a different node within the distributed computing system. Further, one or more elements of the aforementioned control system 140 can be located at a remote location and connected to the other elements over a network.

Software instructions in the form of computer readable program code to perform embodiments of the disclosure can be stored, in whole or in part, temporarily or permanently, on a non-transitory computer readable medium such as a CD, DVD, storage device, a diskette, a tape, flash memory, physical memory, or any other computer readable storage medium. Specifically, the software instructions can correspond to computer readable program code that, when executed by a processor(s) (e.g., processor 150), is configured to perform embodiments of any of the methods described herein.

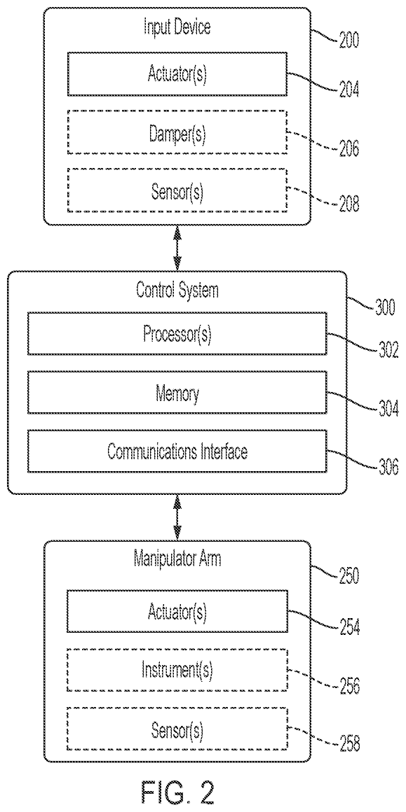

FIG. 2 is block diagram of a computer-assisted system according to some embodiments. As shown in FIG. 2, the computer-assisted system includes an input device 200. The input device may be configured to receive external input from a user. For example, the input device provides a user interface by which a user may impart physical motions and forces to the user interface which are reflected in a following manipulator arm. That is, the manipulator arm may move in accordance with inputs received at the input device based on a predetermined or variable scaling factor, inverse kinematic filter, or other suitable conversion. As shown in FIG. 2, the input device includes actuator(s) 204. The actuator(s) 204 may be configured to apply forces to the input device to provide feedback to a user of the input device. For example, the actuator(s) may be configured to apply forces based on forces sensed at the manipulator arm 250. The actuator(s) may include one more motors, servos, stepper motors, brushless motors, pneumatic actuators, or any other suitable actuator. As shown in FIG. 2, the input device may optionally include physical damper(s) 206. In some embodiments, the dampers may be adjustable dampers such that the amount of damping applied to the input device is controlled by processor(s) according to methods described herein. In some embodiments, the actuator(s) 204 may be commanded to provide damping (e.g., by applying resistive forces opposing movement of the input device in a reference frame of the input device). In some such embodiments, the damping provided by the actuator(s) 204 may be in addition to forces applied based on manipulator arm feedback or other active forces. Actuator-provided damping may be used instead of, or in conjunction with, physical dampers. The input device 200 also includes optional sensor(s) 208. The sensors may utilize any appropriate sensing technology, and may include any appropriate sensor configured to provide information regarding the input device and its operating environment. Example sensors include optical sensors, cameras, accelerometers, inertial measurement units, proximity or contact sensors, force sensors, linear or rotary encoders, potentiometers. In some embodiments, the sensor(s) 208 may include encoders or other joint positions sensors that can provide deflection information from which force information can be computed based on the physical characteristics of input device. In some embodiments, the actuator(s) 204 may provide information to the processor(s) regarding the forces applied to the input device, the position and/or linear velocity, of the input device, and/or the orientation or angular velocity of the input device. “Of the input device” is used here to indicate one or more points or portions of interest of the input device. For example, estimated forces may be determined with an actuator comprising a motor by estimating motor torque from motor current or power, which can be used with the geometry and kinematics of the input device to determine forces applied to the input device. As another example, positions and/or orientations of the input device may be determined with an actuator comprising a motor by monitoring steps of a stepper motor or monitoring a speed of a motor over time. The motor information can be used with the geometry and kinematics of the input device to determine position or velocity information of the input device. Such actuator derived information may be used instead of, or in addition to, sensor information provided by one or more sensors as described above.

According to the embodiment of FIG. 2, a manipulator arm 250 comprises a follower device configured to follow a leader device comprising the input device 200. For example, the manipulator arm 250 may be configured to follow user commands provided by user input at the input device 200. The manipulator arm 250 includes actuator(s) 254. The actuator(s) 254 may be configured to apply forces to portions of the manipulator arm (e.g., arm linkages) to move the manipulator arm 250 and/or an instrument supported by the manipulator arm 250. In some embodiments, the actuator(s) 254 may be configured to apply velocities and/or displacements to portions of the manipulator arm. Motion of the instrument may cause the instrument to apply forces within an operating environment containing the instrument. In some instances, different actuators of the actuator(s) 254 are configured to operate the manipulator arm 250 and an instrument supported by the manipulator arm 250. In some instances, one or more actuators of the actuator(s) 254 are configured to operate both the manipulator arm and one or more instruments supported by the manipulator arm 250. The actuator(s) 254 may be configured to move the manipulator arm 250 based on movements of the input device 200. The actuator(s) 254 may include one or more motors, solenoids, pneumatic or hydraulic pistons, and the like. Example motors include servo motors, stepper motors, brushless motors. As shown in FIG. 2, the manipulator arm may optionally include one or more instruments 256. The one or more instruments may be coupled to the manipulator arm, and in some embodiments may be removable and interchangeable. The manipulator arm 250 also includes optional sensor(s) 258, which may include the example sensors described above with reference to sensor(s) 208. In some embodiments, the sensor(s) may be configured to provide information regarding contact forces on the manipulator arm, position of the manipulator arm, and velocity of the manipulator arm. In some embodiments, the actuator(s) 254 may provide information regarding the forces applied to the manipulator arm as well as the position and/or orientation of the manipulator arm. For example, forces may be measured by converting motor torque and/or current to forces. As another example, positions, orientations, and/or velocities may be measured by monitoring steps of a stepper motor or otherwise monitoring the speed of a motor over time. In some embodiments, the sensor(s) 258 may include force sensors located at a distal portion of an instrument. In some embodiments, the sensor(s) 258 may include one or more strain gauges positioned on a portion of the manipulator arm. A manipulator arm 250 may include any suitable number and type of sensors, as the present disclosure is not so limited.

According to some embodiments as shown in FIG. 2, the computer-assisted system includes a control system 300 which may coordinate and control interoperation between the input device 200 and the manipulator arm 250. The control system 300 may implement methods according to exemplary embodiments described herein for controlling the operation of the input device 200 and the manipulator arm 250. In particular, the control system receives input from the input device 200, and may provide tactile, haptic, and/or force feedback to the input device based on operating characteristics of the manipulator arm 250. The control system also sends commands to the manipulator arm 250 based on the input received at the input device 200 and receives sensor signals from the manipulator arm 250. In some embodiments, the control system may send commands to the manipulator arm based on input received at the manipulator arm 250. For example, the control system may send commands to the manipulator arm to maintain a position when someone pushes on the arm. In some embodiments, the control system may send self-generated commands to the manipulator arm. For example, the control system may send command(s) to perform an automated motion or command(s) to hold position. The control system 300 may command the actuator(s) of both the manipulator arm 250 and the input device 200 in one or more control loops.

As shown in FIG. 2, the control system includes one or more processors 302 which are configured to execute programming instructions that cause the one or more processors to perform exemplary methods described herein. The programming instructions may be stored on memory 304, which may be non-transitory memory. In some embodiments, the control system 300 may cooperate with processor(s) of the input device and manipulator arm to control the input device and manipulator arm. In some embodiments, the control system may physically be implemented in one processor. In other embodiments, the control system may be physically implemented in multiple processors. In some embodiments, a process or the control system may be physically disposed in or on the input device, the manipulator arm, an auxiliary tower, or a user console, or any combination thereof. The control system also includes a communications interface 306 which may facilitate communication of commands, requests, and information between the manipulator arm, input device, and control system. The communications interface 306 can include an integrated circuit for connecting the control system to a network (not shown) (e.g., a local area network (LAN), a wide area network (WAN) such as the Internet, mobile network, or any other type of network) and/or to another device, such as another control system. In some embodiments, the manipulator arm 250 and input device 200 may also include communications interfaces to facilitate communication with the control system 300.

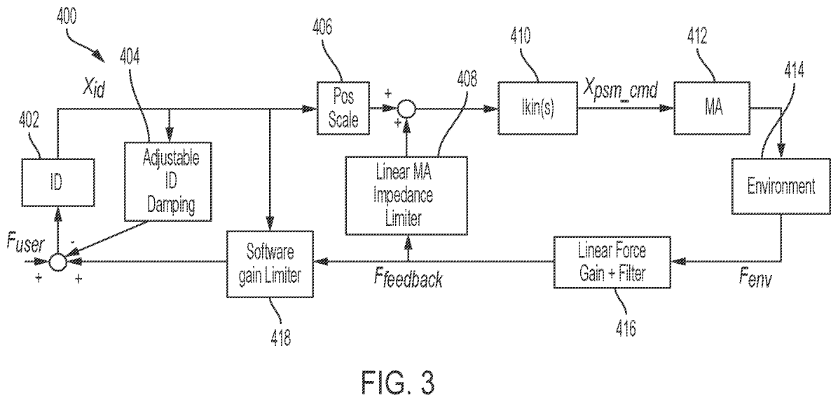

FIG. 3 is a diagram of a control loop 400 for a computer-assisted system according to some embodiments. The control loop shown in FIG. 3 may be representative of control of a computer-assisted system, including an input device and a corresponding manipulator arm. The computer-assisted system may have sub-loops, super-loops, or parallel loops to the control loop shown in FIG. 3, such as for control of specific actuators, specific joints of the manipulator arm or instrument, special functions (e.g., application of energy), other manipulator arms or instruments, other parts of the computer-assisted system, etc. As shown in FIG. 3, the primary input to the control loop 400 is user force input at the input device. The total force acting on the input device including the force from the user may result in the input device motion which is monitored by one or more sensors such as encoders, inertial measurement units and or trackers. That is, the one or more processors of the input device may receive information from one or more sensors and/or actuators of the input device which may be employed to determine a force input from the user and the resultant motion of the input device. In block 402, the control loop outputs a control signal to apply force to the input device via one or more actuators. The control signal may be a function of the force input from the user, an error signal from an adjustable damping in block 404 reducing the force, and a signal from multiple control blocks as discussed further below. The adjustable damping of block 404 may be based on a position change (e.g., velocity) of the input device, and may limit the force rendered to the input device to improve stability. The control of the input device in block 402 may include control of multiple actuators to apply feedback forces to the input device in one or more degrees of freedom of the input device.

As shown in FIG. 3, a signal from the input device (e.g., a position of the input device x_{id}) is provided to block 406, where the position is scaled. As discussed previously, the scaling may include compensating for differences in the size of the input device and the manipulator arm. The scaled position from the input device is then provided to an inverse kinematics block 410. The inverse kinematics block may be configured to receive the scaled position input from the input device and convert that scaled position input into control signals to command one or more actuators to move the manipulator arm toward the target position. In some embodiments, the inverse kinematics block may receive input from the input device and convert this input from input device joint space to three-dimensional space (e.g., Cartesian space) using forward kinematics. The inverse kinematics block may transform the converted input in three-dimensional space to provide a command for the manipulator arm distal portion or support instrument in the three-dimensional space (e.g., Cartesian space). The inverse kinematics block may then convert the command for the manipulator arm to joint space for the manipulator arm using inverse kinematics of the manipulator arm (and as appropriate a supported instrument). The inverse kinematics block 410 may be employed in cases where the kinematics of the input device and manipulator arm are different. As shown in FIG. 3, the control signals from the inverse kinematics block 410 are provided to the manipulator arm in block 412. Block 412 may be representative of an output at the manipulator arm, causing the manipulator arm to follow the inputs at the input device. As shown in FIG. 3, in some embodiments a linear manipulator arm impedance filter (shown in block 408) may be employed as an input to the kinematic filter. The impedance filter may improve stability of the manipulator arm by increasing the compliance of the manipulator arm, which in turn may improve stability of the control loop 400. The linear impedance filter may be based on feedback from the manipulator arm and is optional.

As shown in FIG. 3, following the output to the manipulator arm in block 412, the manipulator arm may in turn output a force and/or motion relative to the operating environment 414. The interaction between the manipulator arm and the operating environment may generate a feedback signal. Namely, as shown in FIG. 3, such interaction may yield an environmental interaction force Fₑₙᵥ. This force signal may be filtered or scaled by a gain factor in block 416. Such scaling and filtering may reduce noise or normalize the forces at the manipulator arm to forces comparable to those employed at the input device (e.g., on the same order of magnitude as the forces employed at the input device). While in FIG. 3 block 416 includes scaling and filtering, in other embodiments scaling and filtering may be performed in distinct blocks. In some embodiments, only one of scaling and filtering may be employed. In some embodiments, scaling and filtering may not be employed. After the application of the gain factor and filtering of block 416, the modified force signal may be representative of a force feedback to be provided to the input device. The modified force signal may also be further adjusted by the impedance limiter of block 408 to determine the force feedback to be provided to the input device. As shown in FIG. 3, the modified force signal may be passed to a software gain limiter in block 418. The software gain limiter may be representative of exemplary embodiments described herein. That is, the software gain limiter may reduce or alter the feedback force provided to the input device based on the modified force signal to maintain stability of the input device when it is actively driven. This software gain limiter may eliminate runaway gain instabilities in the overall control loop 400, as will be discussed further below.

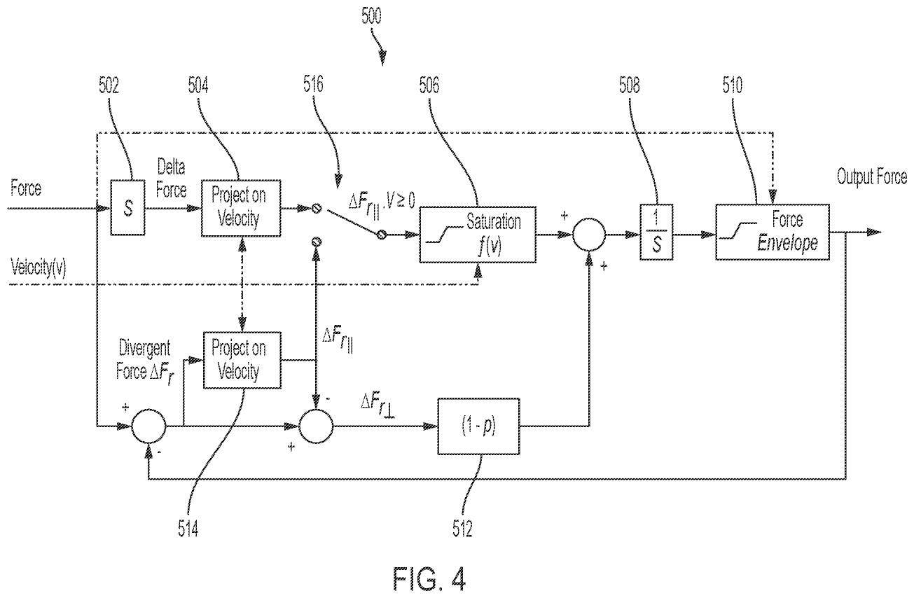

FIG. 4 is a diagram of a control block 500 for a computer-assisted system according to some embodiments. In some embodiments, the control block 500 of FIG. 4 may be representative of the software gain limiter in block 418 of FIG. 3. The control block of FIG. 4 is configured to limit force feedback provided to an input device. As shown in FIG. 4, force and velocity are inputs to the control block. The force input may be scaled and/or filtered force from a manipulator arm. That is, the force input may be a feedback signal from an associated manipulator arm. The velocity input may be measured at the input device and optionally can be scaled and or filtered. According to the embodiment of FIG. 4, several functions of the control block 500 may be dependent on the velocity of the input device, as will be discussed further below.

As shown in FIG. 4, the force is first input to block 502, where a derivative of the force is taken to obtain a delta force (e.g., a change in force). In the embodiment of FIG. 4, the derivative is respective to time. In some embodiments, the derivative of the force may be taken by comparing discrete force measurements over time to obtain the delta force. The delta force is passed to block 504, where the delta force may be projected on velocity. The projection on velocity may include determining a component of the delta force that corresponds to a degree of freedom of the input device. The projection of velocity may employ three dimensional vectors provided by the velocity signal from the input device. In some embodiments, such a projection may be accomplished using a dot product to obtain a matrix of delta forces which correspond to velocities. In some embodiments, the change in force may also be a matrix of multiple forces which may correspond to different degrees of freedom of a manipulator arm. In some embodiments, the degrees of freedom of an input device and a manipulator arm may be the same, such that the force and velocity matrices are the same size. For example, in an input device and manipulator arm having three translational degrees of freedom, the force may be a matrix having three values, each corresponding to a different degree of freedom, and the velocity may be a matrix also having three values, each corresponding to a different degree of freedom. Matrices of any suitable size representative of one or more degrees of freedom may be employed, as the present disclosure is not so limited. According to the embodiment of FIG. 4, the projection on velocity in block 504 functions as a gate to eliminate rendering of force change on the input device on any direction in which the input device has zero velocity.

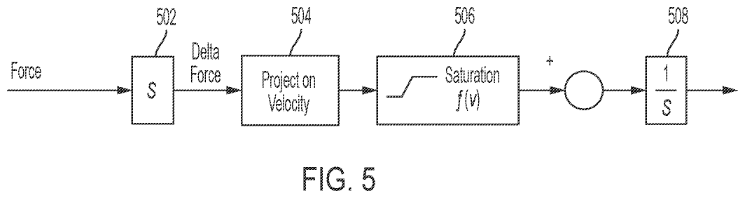

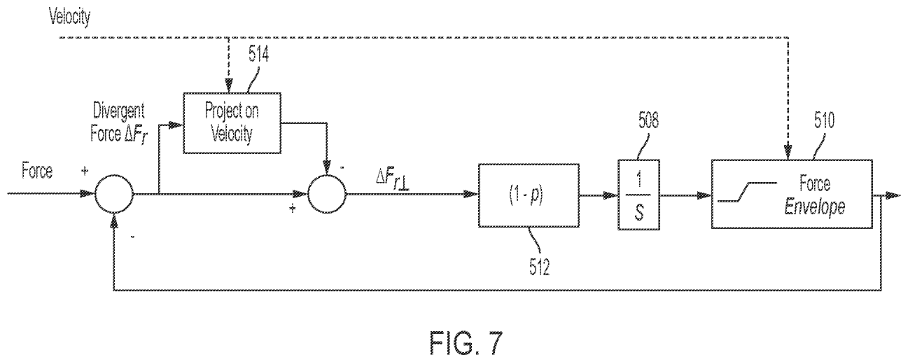

As shown in FIG. 4, after the delta force is projected onto velocity in block 504, it is passed through a ratcheting switch 516 to be discussed further below to a saturation block 506. The saturation block may employ a variable saturation limit based on velocity of the input device, as will be discussed further below with reference to FIG. 5. The saturation function operates effectively as either a pass-through or gain reduction, depending on an environmental stiffness determined between a manipulator arm and its operating environment as well as the environmental stiffness. After applying the saturation function of block 506, the resultant delta force is accumulated (e.g., integrated) in block 508. The integrated force in then passed through a force envelope block 510, discussed further below with reference to FIG. 7. The force envelope block 510 may ensure the force output to the input device does not exceed that measured at the manipulator arm. Force is output from block 510, and the output signal is also provided as an error signal to a portion of the control block.

As shown in FIG. 4, the lower portion of the control loop receives the original force input as well as the previous cycle's (previous cycle of the same control loop) output force from the control block. A difference of these forces is taken to obtain a divergent force. This divergent force is then projected on velocity in block 514, similar to the projection of block 504. In a lower path from block 514, the divergent force that is parallel to directions of motion of the input device (e.g., corresponding to non-zero velocities) is subtracted from the previously obtained divergent force to obtain only force that is perpendicular to any direction having non-zero velocity. This perpendicular divergent force is passed to block 512, which is a temporal recovery block. This block allows the perpendicular divergent force to be superimposed on the force output from the saturation function, which eliminates steady state error over time. In an upper path from block 514, the parallel divergent force is provided to the ratcheting switch 516. The ratcheting switch uses as input the larger force from either block 504 or block 514, which allows the control loop to use excess space in the saturation block 506 to apply feedback forces without generating instabilities. As shown in FIG. 4, the ratcheting switch may determine if the parallel divergent force is positive with respect to the velocity of the input device.

Sub-components and methods of the overall control block of FIG. 4 and their benefits are discussed further below with reference to FIGS. 5-9. It should be noted that these various sub-components may be optional, and in some embodiments may not be employed in a control loop depending on the particular performance characteristics used for feedback forces at an input device.

FIG. 5 is a diagram of a portion of a control block according to some embodiments. As shown in FIG. 5, force is first input to block 502, where a derivative of the force is taken to obtain a delta force (e.g., a change in force). The delta force is passed to block 504, where the delta force may be projected on velocity. As discussed previously, the projection on velocity may include determining a component of the delta force that corresponds to a degree of freedom of an input device. The projection of velocity may employ three dimensional vectors, or any other appropriate number of dimensions, provided by a velocity signal from the input device. According to the embodiment of FIG. 5, the projection on velocity in block 504 functions as a gate to eliminate rendering of force change on the input device on any direction in which the input device has zero velocity. That is, any force change corresponding to a zero velocity will be eliminated or effectively eliminated through the multiplication by zero. This in effect disregards forces in directions not correlated with a direction of motion of the input device for determination of feedback force.

As shown in FIG. 5, after the delta force is projected onto velocity in block 504, it is passed to a saturation block 506. The saturation block may employ a variable saturation limit based on velocity of the input device as well as an environmental stiffness determined from information at a manipulator arm. The saturation function operates effectively as either a pass-through or gain reduction, depending on an environmental stiffness determined between a manipulator arm and its operating environment as well as the environmental stiffness. In some embodiments, a saturation limit of the saturation function may be defined as:

$$Saturation Limit = {K}_{max} * {v}_{ID} * Δ t$$

Where v_{ID} is the magnitude of velocity of the input device, Kₘₐₓ is a stiffness threshold (e.g., a maximum stiffness threshold) determined on the particular kinematics of a manipulator arm and input device, and Δt is a time interval used to control the manipulator arm control system and may be used as an integration time where appropriate in the control loop. The saturation function caps its output to this saturation limit. If the force or environmental stiffness are low enough, the saturation function operates as a passthrough. In cases where the environmental stiffness is greater than the stiffness threshold, the saturation function may operate as a gain reduction for the force change. The environmental stiffness may be determined by comparing change in force at a manipulator device instrument and its change in position. After applying the saturation function of block 506, the resultant delta force is accumulated (e.g., integrated) in block 508. After accumulation in block 508, the force may be output to an input device.

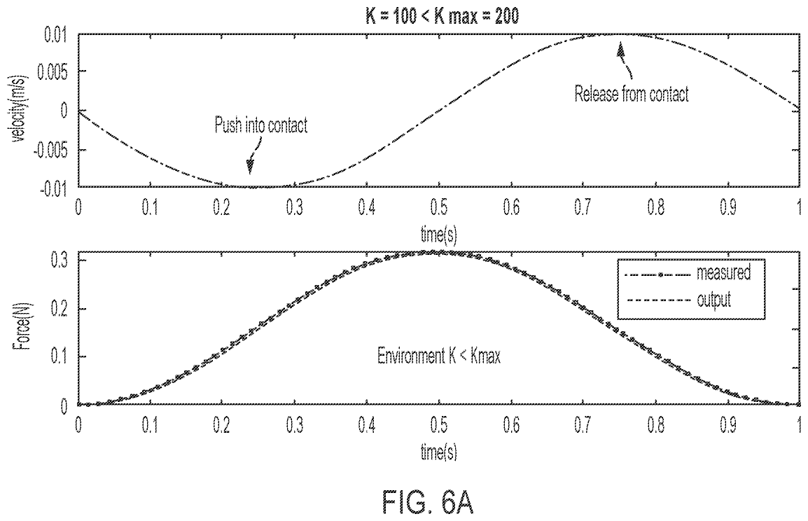

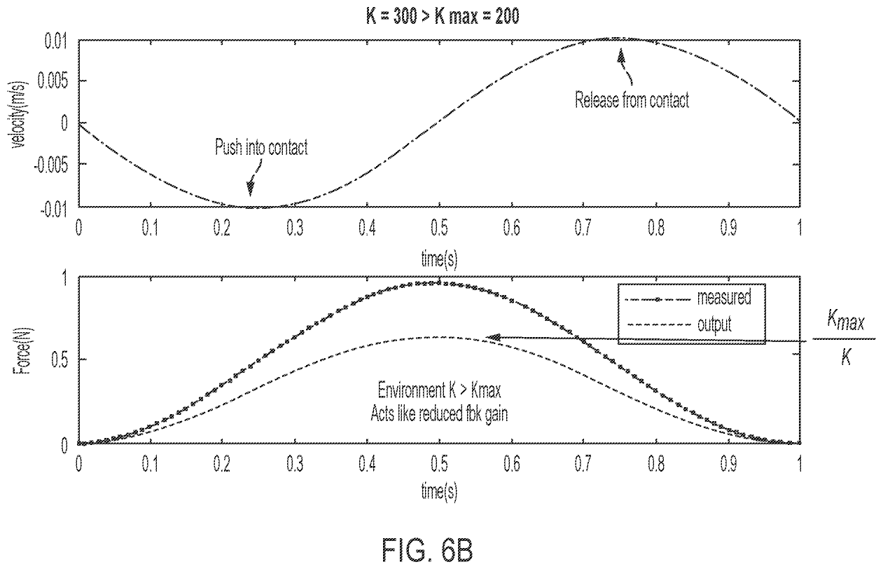

FIG. 6A depicts experimental results of an implementation of the control block of FIG. 5 in simulation. As shown in FIG. 6A, the environmental stiffness K is 100, which is less than a stiffness threshold of 200. Accordingly, when an instrument is pushed into contact with a surface or within the bulk of an object or other material, and when the instrument is released from contact with that surface or from that part of the bulk of the object or other material, the forces generated may be rendered in “full” back to an input device. “Full rendering” is used here to indicate what would be provided if saturation limits were not applied-full rendering may be scaled in accordance with a scaling relationship between the instrument operating environment and the input device operating environment. The full rendering of force in this example is because the saturation limit of the saturation function is not reached due to the lower environmental stiffness. Accordingly, in this example the saturation function operates as a pass-through, allowing the feedback force to be applied to the input device in full.

FIG. 6B depicts experimental results of an implementation of the control block of FIG. 5 in simulation. As shown in FIG. 6B, the environmental stiffness K is 300, greater than the stiffness threshold Kₘₐₓ of 200. Accordingly, when an instrument is pushed into contact with an environmental surface, or into a bulk or other material, and released from the contact with that surface, or from that part of the bulk or other material, the feedback forces determined according to the control block of FIG. 5 are reduced compared to those measured at the manipulator arm. Because the environmental stiffness is greater and the stiffness threshold, the saturation limit is reached and is not exceeded. Accordingly, in the case the stiffness threshold is exceeded by the environmental stiffness, the saturation function operates as a fixed gain reduction based on a comparison (e.g., a difference or ratio) between the threshold stiffness and the environmental stiffness. In some embodiments, the comparison may be compared individually compared as scalars in multiple degrees of freedom. That is, a stiffness comparison may be performed for each degree of freedom of the instrument and associated input device. In the example shown in FIGS. 6A-6B, a single degree of freedom is shown for simplicity. For example, in some embodiments, the saturation function may employ the ratio of threshold stiffness to environmental stiffness Kₘₐₓ/K to determine the change in output feedback force. That is, the control block may determine a gain factor based at least in part on the threshold stiffness and the environmental stiffness and apply that gain factor to the change in force output to the input device where in this embodiment the above ratio may be applied as the gain factor. As another example, the saturation function may change (e.g., reduce) the output feedback force based on the difference in stiffness between an environmental stiffness and the threshold stiffness when the environmental stiffness is larger than threshold stiffness. For example, the saturation function may determine a force reduction according to the following equation when the environmental stiffness is larger than the threshold stiffness:

$$Force Reduction = ( K - {K}_{max} ) * {v}_{ID} * Δ t$$

In still other embodiments a saturation function may employ any linear or non-linear operation to alter (e.g., reduce) the output feedback force based on the environmental stiffness and threshold stiffness.

FIG. 7 is a diagram of a portion of a control block according to some embodiments. The control block of FIG. 7 may optionally be employed with other exemplary embodiments herein (e.g., the embodiment of FIG. 5) to reduce the effects of steady state error and/or to address force discrepancies in cases of no input device velocity in the direction of steady state error. As shown in FIG. 7, the control block receives a force input from a manipulator arm as well as the previous cycle's output force from the control block. A difference of these forces is taken to obtain a divergent force. This divergent force is then projected on velocity in block 514, which uses a velocity input signal from an input device. The divergent force that is parallel to directions of motion of the input device (e.g., corresponding to non-zero velocities) is subtracted from the previously obtained divergent force to obtain only force that is perpendicular to direction of velocity. This perpendicular divergent force is passed to block 512, which is a temporal recovery block. This block allows the perpendicular divergent force (e.g., a restoring force) to be applied to the input device over time, which eliminates steady state error over time. In the embodiment of FIG. 7, the temporal recovery block may implement exponential decay. That is, the divergent force applied to the input device will increase in an exponential decay profile until the divergent force is fully applied. In other embodiments the temporal recovery may implement a linear force recovery or any other suitable force profile, as the present disclosure is not so limited. In some embodiments, the temporal recovery block may implement a time delay before applying the divergent force to the input device. For example, the control block may determine if a threshold time period of steady force has been exceeded before adding the divergent force to the input device.

It should be noted that the restoring divergent force applied by the control block of FIG. 7 may not introduce instabilities in an input device, because in the embodiment of FIG. 7 the forces are only applied in directions perpendicular to a velocity direction. A result of this limitation is that force will not be applied to the input device if it causes the input device to move, as the force would immediately generate a non-zero velocity in that direction. Accordingly, the restoring force provided by the embodiment of FIG. 7 may be applicable in situations where a user is holding an input device steady and resisting forces in one or more directions.

As shown in FIG. 7, the output from the temporal recovery block 512 is provided to an integration block 508, which accumulates the divergent force for application to the input device. In some embodiments as shown in FIG. 7, the control block also includes a force envelope block 510. The force envelope block is configured to limit the force output at the input device to be less than or equal to a scaled force measure at an instrument of a manipulator device, regardless of velocity. For example, in cases where a change in force reducing the magnitude is not applied as a feedback force to the input device because velocity of the input device zero, the force envelope block would capture that reducing change in force and correspondingly reduce the force output of the input device. Such a block may be beneficial in alerting a user of the computer-assisted system that there has been a loss in force at an instrument even if the input device is not moving and being held stationary.

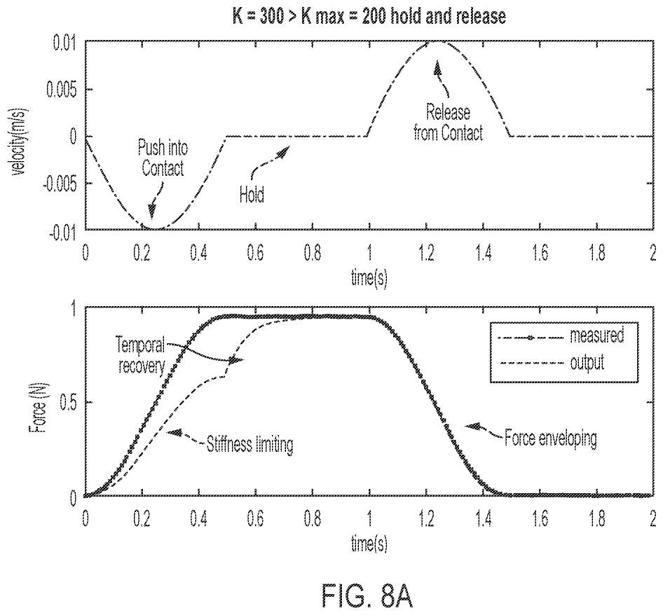

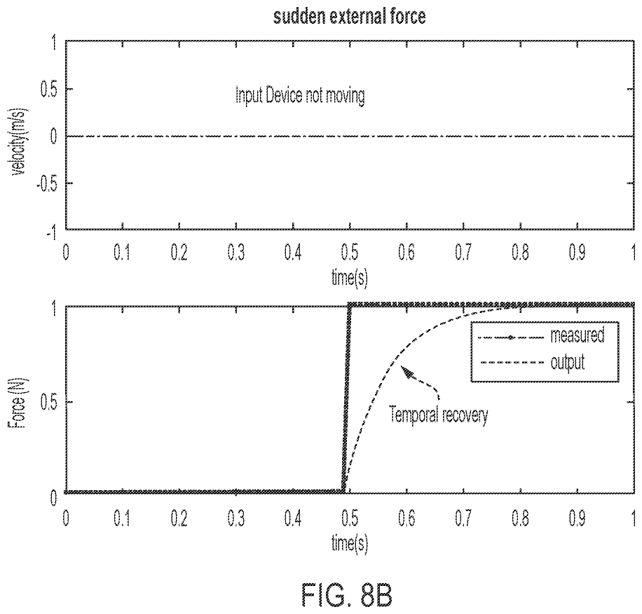

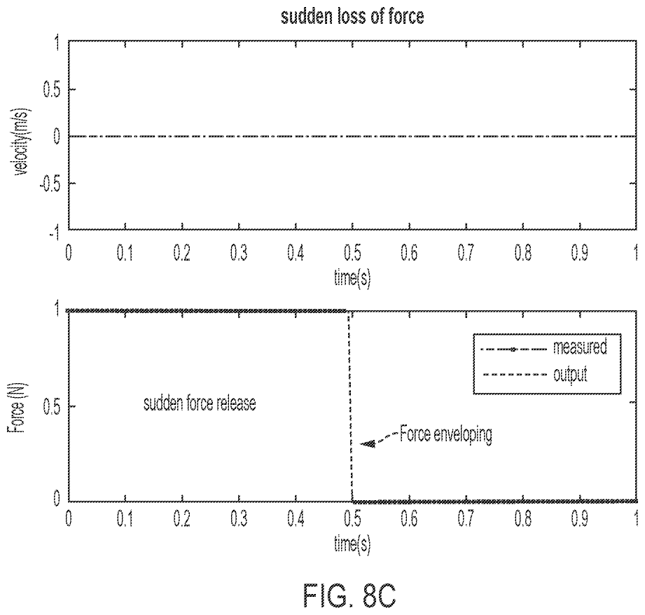

FIG. 8A depicts experimental results of an implementation of the control block of FIG. 7 in simulation. FIG. 8A shows a situation where an instrument is pushed into contact with its operating environment (e.g., surface, bulk, etc.), held in place, and then released from contact. Additionally, an environmental stiffness is greater than a threshold stiffness, so a saturation function (see FIG. 5) or other stability control function acts as a gain reduction that may cause steady state error in a feedback force rendered to the user at an input device. As shown in FIG. 8A, the force is limited until a temporal recovery block operates to provide a restoring force to the input device. As the velocity in the direction corresponding to this example is zero, the force is able to be applied in an exponential decay profile. This situation may correspond to a user holding the input device stationary. When the instrument is released from contact, a force enveloping function ensures the output force is less than or equal to the measured force, regardless of input device velocity. In this manner, the control block of FIG. 8A may improve user feel by improving the accuracy of force feedback without introducing additional instabilities into the input device.

FIG. 8B depicts experimental results of an implementation of the control block of FIG. 7 in simulation. In the example of FIG. 8B, the input device is not moving and has a constant zero velocity. However, a sudden external force is applied to an instrument whereas prior there was no force applied to the instrument. For example, another instrument may contact the instrument, or a portion of the operating environment may move, thereby imparting a force on the instrument. In some embodiments, this force would not be rendered to the input device because of the zero velocity of the input device. However, a temporal recovery restoring force as shown in FIG. 7 may be employed to render the force to the input device so long as the input device remains at zero velocity. Accordingly, force may be accurately rendered in cases where the input device is held stationary. If the input device was free to move, the application of force would cause the input device to correspondingly move, which would cancel further application of force to the input device in that direction.

FIG. 8C depicts experimental results of an implementation of the control block of FIG. 7 in simulation. In the example of FIG. 8C, the input device is not moving and has a constant zero velocity. Additionally, there is a constant force at an instrument, which may be the case when an instrument is held in contact with its operating environment or carrying a fixed weight. The force is suddenly released while the velocity of the input device remains zero. For example, the operating environment in contact with the instrument may move or yield, causing a loss in force. In some embodiments, this force would not be rendered to the input device because of the zero velocity. However, force enveloping as shown in FIG. 7 may be employed to reduce the force applied to the input device to be less than or equal to the force measured at the instrument. The force enveloping may account for differences in scale between the input device and instrument when limiting the force. Accordingly, force may be accurately rendered in cases where the input device is held stationary and there is a loss of force.

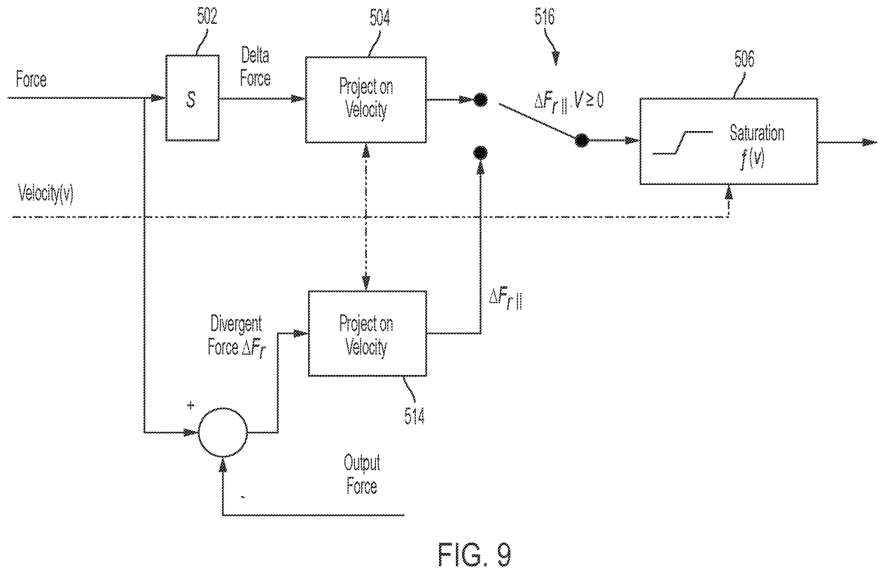

FIG. 9 is a diagram of a portion of a control block according to some embodiments. As shown in FIG. 9, the control block receives a force input from a manipulator arm as well as the previous output force from a control loop cycle. The force input is differentiated in block 502 and projected on a velocity of the input device in block 504. A ratcheting switch 516 is employed to allow the control block to maximize use of a saturation function in block 506. That is, the ratcheting switch is configured to pass additional force into the saturation function when certain conditions are met to retain stability. The saturation function in block 506 may operate similarly to that described with reference to FIG. 5. A difference of these forces is taken to obtain a divergent force. As shown in FIG. 9, the output force from a prior cycle is subtracted from the force input to obtain a divergent force. This divergent force is then projected on velocity in block 514 to determine components of the divergent force corresponding to input device velocity, similar to the projection of block 504. From block 514, the parallel divergent force is provided to the ratcheting switch 516. The ratcheting switch uses as input the larger force from either block 504 or block 514, which allows the control block to use excess space in the saturation block 506 to apply feedback forces without generating instabilities. As shown in FIG. 9, the ratcheting switch may determine if the parallel divergent force is positive with respect to the velocity of the input device. The ratcheting switch may determine if the divergent change is force is in an opposite direction of the velocity of the input device, and if that condition is satisfied, may activate the switch to use the divergent switch as an input to the saturation function. Such an arrangement ensures the larger force is input to saturation function in block 506 under the condition where the change in force is opposite to the direction of velocity of the input device.

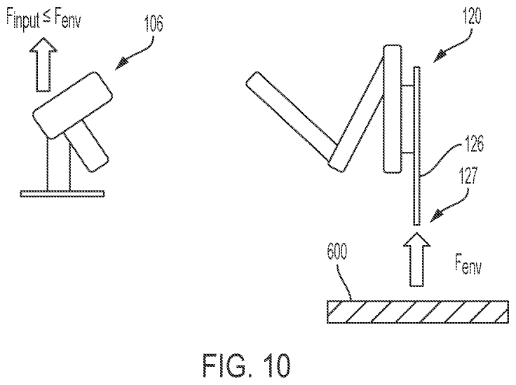

FIG. 10 is a simplified diagram of a computer-assisted system during operation according to some embodiments. As shown in FIG. 10, the computer-assisted system includes an input device 106 and a manipulator arm 120. The manipulator arm includes an instrument 126 having a distal portion 127 (e.g., a distal end) which physically interacts with an operating environment 600. The operating environment may include objects (e.g., tables, working surfaces, components, etc.), surfaces (e.g., ground, wall, ceiling), or subjects (e.g., patients), depending on the particular application. As discussed previously, the input device 106 and the manipulator arm 120 may be remotely located from another other and may communicate via a control system configured to coordinate the operation of the input device and manipulator arm. The manipulator arm may be configured to proportionally follow movements and forces applied by a user to the input device. That is, user input received at the input device may be the basis for commands sent to the manipulator arm to move and interact with the operating environment 600.

According to the embodiment of FIG. 10, the computer-assisted system may be controlled to provide feedback forces to the input device 106. Force may be measured at the instrument between the operating environment and the instrument in one or more directions, represented by Fₑₙᵥ. This force may be employed to provide a feedback force to the input device 106, after appropriate scaling, filtering, and/or limiting according to exemplary embodiments herein. In some embodiments, the force may be measured at the instrument in at least two degrees of freedom (e.g., three degrees of freedom) and may be employed to generate feedback forces in at least two corresponding degrees of freedom of the input device. In some embodiments, the force may be measured in a single degree of freedom and may be employed to generate feedback force in a corresponding single degree of freedom. For example, forces in the same Cartesian direction based on a local reference frame may be measured at the instrument and generated at the input device. As shown in FIG. 10, the feedback force at the input device may be limited according to a force enveloping function, which limits the scaled force at the input device, Fᵢₙₚᵤₜ, to be less than or equal to the force at the instrument after accounting for scaling. The force envelopment may be determined independently in the at least two degrees of freedom. The enveloping block simply operates by comparing the input force of 510 (F_{env_in}) with the input force of 502 (Fᵢₙ). When the dot product of the input force of 502 and input force of 510 is negative, the output force of 510 (Fₒᵤₜ) is zeroed out, otherwise the output force of 510 is in same direction as the input force of 510, with its magnitude saturated by the magnitude of the input force of 502 projected on to the input force of 510.

$$if {F}_{i n} . {F}_{env \_ in} < 0$$

$$limit = 0$$

$$else$$

$$limit = \vert {proj}_{{F}_{env \_ in}} {F}_{i n} \vert$$

$${F}_{out} = Bound ( {F}_{env \_ in} , limit )$$

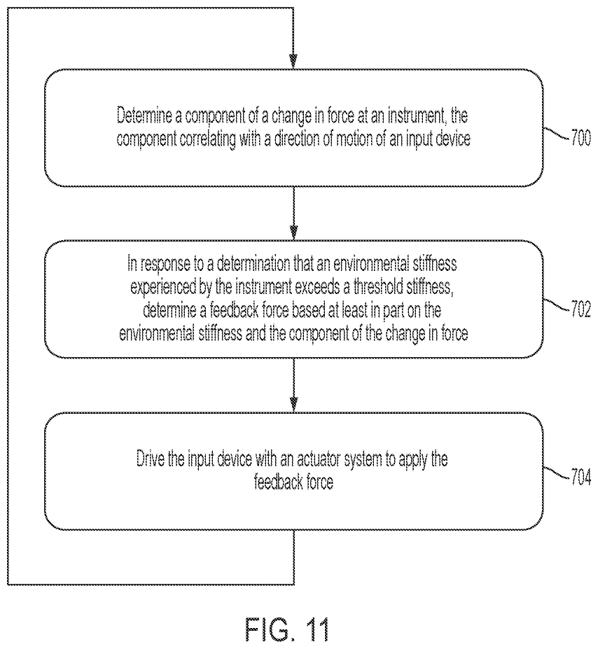

FIG. 11 depicts a flow chart for a method of operating a computer-assisted system according to some embodiments. In block 700, a component of a change in force at an instrument that correlates with a direction of motion of an input device is determined. For example, one or more force sensors, strain gauges, or other appropriate sensors may be employed to determine force at the instrument, and that force may be projected onto a velocity of the input device in one or more degrees of freedom. The method may also include determining an environmental stiffness of the instrument. For example, the change in force measured at the instrument may be compared to a change in displacement of the instrument to determine an environmental stiffness. In block 702, in response to a determination that an environmental stiffness experienced by the instrument exceeds a threshold stiffness, the method includes determining a feedback force based at least in part on the environmental stiffness and the component of the change in force. In some embodiments, the threshold stiffness may be a maximum stiffness used to set a saturation limit of a saturation function. In some embodiments, determining the feedback force may be based on a gain factor that is determined by the environmental stiffness and threshold stiffness. In some embodiments, determining the feedback force may also include one or more additional methods as described herein. For example, determining the feedback force may include one or more of force envelopment, a ratcheting switch, and a temporal recovery restoring force determination as described herein. In block 704, the input device is driven with one or more actuators of an actuator system to apply the determined feedback force.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

The terms “program” or “software” are used herein in a generic sense to refer to any type of computer code or set of computer-executable instructions that can be employed to program a computer or other processor to implement various aspects of the present disclosure as discussed above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A computer-assisted system comprising:
a manipulator arm configured to support an instrument;
an input device configured to accept user commands to move the instrument;
an actuator system coupled to the input device; and
a controller comprising at least one processor, the controller configured to:
determine a component of a change in force at the instrument, the component correlating with a direction of motion of the input device,
in response to a determination that an environmental stiffness experienced by the instrument exceeds a threshold stiffness, determine a feedback force based at least in part on the environmental stiffness and the component of the change in force, and
cause the actuator system to drive the input device to apply the feedback force.

2. The computer-assisted system of claim 1, wherein the instrument comprises a distal portion, and wherein the change in force comprises a change in distal force at the distal portion of the instrument.

3. The computer-assisted system of claim 1, further comprising:
one or more sensors coupled to the manipulator arm, the one or more sensors configured to sense forces applied to the manipulator arm,
wherein the controller is configured to determine the change in force based on the forces applied to the manipulator arm.

4. The computer-assisted system of claim 1, wherein the direction of motion of the input device is determined in at least two degrees of freedom, and wherein the direction of motion of the input device is a direction of a velocity of the input device.

5. The computer-assisted system of claim 1, wherein the controller is configured to determine the feedback force by:
determining the feedback force as opposing the direction of motion of the input device.

6. The computer-assisted system of claim 1, wherein the controller is configured to determine the feedback force based at least in part on the environmental stiffness and the threshold stiffness by:
using a comparison of the environmental stiffness and the threshold stiffness.

7. The computer-assisted system of claim 1, wherein the controller is configured to determine the feedback force based at least in part on the environmental stiffness and the threshold stiffness by:
determining a gain factor based at least in part on the threshold stiffness and environmental stiffness, and
applying the gain factor to the component of the change in force.

8. The computer-assisted system of claim 7, wherein determining the gain factor comprises:
using a ratio between the threshold stiffness and the environmental stiffness; or
using a difference between the threshold stiffness and the environmental stiffness.

9. The computer-assisted system of claim 1, wherein the controller is further configured to:
determine a saturation limit based on the environmental stiffness and the threshold stiffness, and
apply the saturation limit to the component of the change in force to produce a resultant change in force,
wherein the controller is configured to determine the feedback force by accumulating the resultant change in force.

10. The computer-assisted system of claim 1, wherein the controller is configured to determine the feedback force by:
limiting the feedback force to a force limit determined based on a force at the instrument.

11. The computer-assisted system of claim 10, wherein the controller is further configured to:
determine the force limit in at least two degrees of freedom of the input device, the at least two degrees of freedom of the input device correlating with at least two degrees of freedom of the instrument.

12. The computer-assisted system of claim 1, wherein the controller is configured to determine the feedback force by:
using only the component of the change in force correlating with the direction of motion of the input device and not any other components of the change in force, or
disregarding any components of the change in force not correlating with the direction of motion of the input device to determine the feedback force.

13. The computer-assisted system of claim 1, wherein the controller is further configured to:
cause the actuator system to drive the input device to apply a restoring force superimposed on the feedback force, the restoring force being in a direction perpendicular to the direction of motion of the input device.

14. The computer-assisted system of claim 13, wherein the controller is further configured to:
determine an instrument force at the instrument, and
increase the restoring force over time until a total force applied by the input device is no less than the instrument force.

15. The computer-assisted system of claim 1, wherein the controller is further configured to, in response to a determination that the change in force plus an error in the feedback force is greater than a previous change in force at the instrument:
determine if the change in force has an opposite component correlating with an opposite direction of the direction of motion of the input device, and
in response to determining that the change in force has the opposite component,
determine the feedback force further based in the opposite component plus the error.

16. The computer-assisted system of claim 1, wherein:
the actuator system is a first actuator system;
the computer-assisted system further comprises a second actuator system coupled to the manipulator arm, the second actuator system configured to drive motion of the instrument; and
the controller is further configured to:
determine the environmental stiffness based on an amount of force or torque associated with the second actuator system when driving the motion of the instrument, or
determine the environmental stiffness based on a resulting motion of the instrument when driving the motion of the instrument with the second actuator system.

17. The computer-assisted system of claim 1, wherein: the actuator system is a first actuator system;
the computer-assisted system further comprises a second actuator system coupled to the manipulator arm; and
the controller is further configured to:
cause the second actuator system to drive motion of the instrument in response to an operator input received at the input device, and
determine the environmental stiffness based on a motion of a point when the second actuator system is driving the motion of the instrument, wherein the point is fixed relative to the instrument.

18. A method of controlling a computer-assisted system configured to support an instrument, the computer-assisted system comprising an input device configured to control movement of the instrument and an actuator system configured to apply force to the input device, the method comprising:
determining a component of a change in force at the instrument, the component correlating with a direction of motion of the input device;
in response to a determination that an environmental stiffness experienced by the instrument exceeds a threshold stiffness, determining a feedback force based at least in part on the environmental stiffness and the component of the change in force; and
causing the actuator system to drive the input device to apply the feedback force.

19. The method of claim 18, wherein the instrument comprises a distal portion, and wherein the change in force comprises a change in distal force at the distal portion of the instrument.

20-32. (canceled)

33. A non-transitory computer-readable storage medium storing instructions that, when executed by at least one processor associated with a computer-assisted device, causes the at least one processor to perform operations,
wherein the computer-assisted device comprises:
a manipulator arm configured to support an instrument,
an input device configured to accept user commands to move the instrument, and
an actuator system coupled to the input device;
wherein the operations comprise:
determining a component of a change in force at the instrument, the component correlating with a direction of motion of the input device,
in response to a determination that an environmental stiffness experienced by the instrument exceeds a threshold stiffness, determine a feedback force based at least in part on the environmental stiffness and the component of the change in force, and
cause the actuator system to drive the input device to apply the feedback force.
